(19) United States
(12) Patent Application Publication
(10) Pub. No.: US 2014/0364351 A1
(43) Pub. Date: Dec. 11, 2014

(54) Process for preparing a mixture of alcohols

(71) Applicant: RHODIA OPERATIONS, Aubervilliers (FR)

(72) Inventors: Virginie Belliere-Baca, Andresy (FR); Didier Morvan, Mornant (FR); Loïc Baussaron, Venissieux (FR); Gérald Bocquenet, Communay (FR); Roland Jacquot, Francheville (FR); Philippe Marion, Vernaison (FR); Jun Kubo, Saitama (JP); Tetsuya Yoshioka, Saitama (JP)

(21) Appl. No.: 14/366,281

(22) PCT Filed: Dec. 20, 2012
(86) PCT No.: PCT/EP2012/076272

§ 371 Date: Jun. 18, 2014

(30) Foreign Application Priority Data
Dec. 20, 2011 (FR) 1162083

(51) Int. Cl. C07C 29/36

(52) U.S. Cl. CPC C07C 29/36
USPC 508/583; 252/364; 252/182.12; 106/311; 44/452

(57) ABSTRACT

A method for preparing a mixture (M) including at least one alcohol (Aj), wherein said method includes a gas-phase oligomerization reaction of at least one alcohol (Ai) with a solid acid-base catalyst doped with one or more metals, said reaction being carried out in the presence of hydrogen and at a temperature of no less than 50° C. and strictly less than 200° C.

The present invention relates to a process for preparing a mixture of alcohols.

Industrially, the most important alcohols are ethanol, 1-propanol, n-butanol, alcohols for plasticizers containing a C6-C11 alkyl chain and fatty alcohols containing a C12-C18 alkyl chain, used as detergents. These various alcohols are prepared from fossil resources either via an olefin oxidation route or via the Ziegler process (oxidation of trialkylaluminum) (K. Ziegler et al., Justus Liebigs Ann. Chem. 629 (1960) 1). Alcohols are also used as solvents, diluents for paints (mainly light alcohols bearing a C1-C6 alkyl chain), as intermediates leading to esters, but also as organic compounds, as lubricants or as fuels.

The synthesis of these alcohols often involves several steps and leads to mixtures of alcohols. For example, alcohols bearing a C6 alkyl chain are synthesized by co-dimerization of butene and propene, followed by conversion into a mixture of aldehydes by hydroformylation, before being hydrogenated, finally leading to a mixture of alcohols bearing a C6 alkyl chain. For example, butanol has hitherto predominantly been produced via the process of hydroformylation of propylene, a petroleum derivative (Wilkinson et al., Comprehensive Organometallic Chemistry, The Synthesis, Reactions and Structures of Organometallic Compounds, Pergamon Press 1981, 8). Butanol may also be obtained via fermentation processes, which have returned to the forefront as a result of the increase in petroleum raw materials. Acetobutyl fermentation, more commonly known as ABE fermentation, coproduces a mixture of ethanol, acetone and butanol in a weight ratio in the region of 1/3/6. The bacterium that is the source of the fermentation belongs to the family of Clostridium acetobutylicum.

Given the diversity of alcohols required for the chemical industry and the broad range of use, there is therefore a need to develop a simplified process for forming alcohols that leads to good yields and minimizes the mixtures. It is also advantageous to have a flexible process enabling the use of ethanol derived from renewable materials to form heavier biosourced alcohols.

The aim of the present invention is to provide a process comprising a simplification of the step for separating the alcohols formed.

Another aim of the present invention is to provide a process for obtaining a mixture of alcohols that is free of aromatic compounds, such as xylene or benzene, and which has a limited number of species chosen from unsaturated alcohols such as crotonyl alcohols (cis and trans), buten-1-ol, hexenols and alcohologens such as butanal, hexanal or crotonaldehydes (cis and trans).

Furthermore, an aim of the invention is to provide a process for stabilizing the reaction medium.

An aim of the invention is also to provide a process that affords a substantial saving in energy.

Another aim of the present invention is to provide a process for preparing alcohols, and especially butanol, which is easy to perform and which leads to a better overall yield for the reaction.

Furthermore, an aim of the invention is to provide a process making it possible largely to limit the treatment of the streams. Thus, one of the aims of the invention is to provide a simplified process that affords a saving in space devoted to the equipment, and also a gain in time and ease of implementation.

One subject of the present invention is thus a process for preparing a mixture (M) comprising at least one alcohol (Aj), said process comprising a gas-phase oligomerization reaction of at least one alcohol (Ai), performed in the presence of hydrogen, and of a solid acid-base catalyst doped with one or more metals, at a temperature of greater than or equal to 50° C. and strictly less than 200° C.

Preferably, the reaction is performed at a temperature from 80° C. to 195° C., in particular from 100° C. to 195° C., preferentially from 150° C. to 195° C., very preferentially between 170° C. and 195° C. and even more preferentially between 180° C. and 195° C.

In the context of the invention, and unless otherwise mentioned, the term “alcohols (Ai)” means alcohols whose linear or branched alkyl chain comprises n carbon atoms, with n representing an integer from 1 to 10. According to the invention, the term “alcohols (Ai)” also encompasses the term “starting alcohols”. The “alcohols (Ai)” according to the invention may be, for example: methanol, ethanol, propanol, butanol, pentanol, heptanol, hexanol, octanol, nonanol or decanol. The alcohols (Ai) denote the starting alcohols before the oligomerization step.

In the context of the invention, and unless otherwise mentioned, the term “alcohols (Aj)” means alcohols whose linear or branched alkyl chain comprises m carbon atoms, with m representing an integer from 2 to 20. According to the invention, the term “alcohols (Aj)” also encompasses the term “formed alcohols” or “upgradable alcohols”. The “alcohols (Aj)” according to the invention may be, for example, ethanol, propanol, butanol, pentanol, heptanol, hexanol, octanol, decanol, ethyl-2-butanol and ethyl-2-hexanol. According to the invention, the mixture (M) comprises butanol.

In the context of the invention, the alcohols (Aj) are obtained by oligomerization of one or more alcohols (Ai).

In the context of the invention, and unless otherwise mentioned, the term “oligomerization of an alcohol” means a process for transforming an alcohol monomer into an alcohol oligomer. According to the invention, the oligomerization may be, for example, a dimerization.

In the context of the invention, and unless otherwise mentioned, the term “from x to y” means that the limits x and y are included. For example, “an integer from 2 to 20” means that the integer is greater than or equal to 2 and less than or equal to 20.

Preferentially, the alcohol (Ai) is ethanol.

According to a particular embodiment, the oligomerization is a dimerization, preferentially a dimerization of ethanol. In this embodiment, the mixture (M) obtained comprises butanol.

According to a particular embodiment, the present invention relates to a process for is preparing a mixture (M) comprising at least one alcohol (Aj), said process comprising a gas-phase ethanol dimerization reaction, performed in the presence of hydrogen, and of a solid acid-base catalyst doped with one or more metals, at a temperature of greater than or equal to 50° C. and strictly less than 200° C.

According to the invention, the alcohol(s) (Ai) used may be anhydrous or aqueous. If the alcohol(s) (Ai) used are aqueous, they may comprise from 0.005% to 20% by weight of water relative to the total weight of alcohol(s) (Ai).

In the context of the invention, and unless otherwise mentioned, the term “solid acid-base catalyst” means a solid acid-base catalyst that has not been doped. The term “solid acid-base catalyst” also denotes a “solid acid-base catalyst before doping” or an “undoped solid acid-base catalyst”.

In the context of the invention, and unless otherwise mentioned, the term “doped solid acid-base catalyst” means a solid acid-base catalyst as defined above, which has been modified with a dopant, such as one or more metals. Thus, a doped solid acid-base catalyst corresponds to a solid acid-base catalyst as defined above, which has been doped with one or more metals.

According to one aspect of the invention, the solid acid-base catalyst before doping may be chosen from the group consisting of:
alkaline-earth metal phosphates, especially calcium phosphates such as tricalcium phosphates, hydrogen phosphates or hydroxyapatites;
hydrotalcites;
zeolites; and
mixtures of metal oxides.

Thus, according to the invention, the doped solid acid-base catalyst may be chosen from the group consisting of doped alkaline-earth metal phosphates, doped hydrotalcites, doped zeolites and mixtures of doped metal oxides.

According to a particular embodiment, the solid acid-base catalyst before doping is an alkaline-earth metal phosphate, chosen especially from calcium phosphates such as tricalcium phosphates, hydrogen phosphates and hydroxyapatites. Preferably, for all these phosphates, it is possible to use these salts with the stoichiometry Ca₃(PO₄)₂, CaHPO₄ or Ca₁₀(PO₄)₆(OH)₂ or these same non-stoichiometric salts, i.e., with Ca/P molar ratios different from that of their empirical formula, so as to modify the acidity-basicity thereof. In general, these salts may be in crystalline or amorphous form. Some or all of the calcium atoms may be replaced with other alkaline-earth metal atoms without this harming the performance qualities of the final catalyst.

According to another embodiment, the solid acid-base catalyst before doping is chosen from hydrotalcites. Hydrotalcites or lamellar double hydroxides may have a general formula M²⁺₁₋ₓM³⁺ₓ(OH)₂(Aⁿ⁻_{x/n}).yH₂O, M²⁺ being a divalent metal and M³⁺ a trivalent metal; A is either CO₃²⁻ in which n=2, or OH⁻ in which n=1; x is from 0.66 to 0.1 and y is from 0 to 4. Preferably, the divalent metal is magnesium and the trivalent metal is aluminum. In the latter case, the empirical formula may be Mg₆Al₂(CO₃)(OH)₁₆.4H₂O. According to the invention, a modification of the ratio M³⁺/M²⁺ is possible while at the same time maintaining the hydrotalcite structure, which makes it possible to modulate the acidity-basicity of the catalytic support. Another way of modifying the acidity-basicity of this family of supports may be to replace the divalent metal with another metal of identical valency, the same substitution operation being possible with the trivalent metal.

According to another embodiment, the solid acid-base catalyst before doping is chosen from zeolites. According to the invention, the zeolites are not in their acidic form, but in their sodium form, in which some or all of the sodium ions may be exchanged with other alkali metals or alkaline-earth metals (LiX, LiNaX, KX, X being an anion, for example a halide anion such as chloride). These catalysts may be prepared by cation exchange using zeolites in sodium form and a solution containing the cations to be introduced in the form of a water-soluble salt, such as chlorides or nitrates.

According to another embodiment, the solid acid-base catalyst before doping is chosen from mixtures of metal oxides, especially binary mixtures of metal oxides such as ZnO and Al₂O₃, SnO and Al₂O₃, Ta₂O₅ and SiO₂, Sb₂O₅ and SiO₂, MgO and SiO₂, or Cs₂O and SiO₂, so as to obtain a support with bifunctional properties. Ternary mixtures of metal oxides may also be used, such as MgO/SiO₂/Al₂O₃. Depending on the reaction conditions, the ratio of the two oxides present in a binary mixture may be modified as a function of the specific surface areas and of the strength of the acidic and basic sites.

According to a particular embodiment, the solid acid-base catalyst before doping is of alkaline-earth metal phosphate type, especially calcium phosphate.

Preferably, the solid acid-base catalyst before doping is chosen from calcium hydroxyapatites. In this case, the doped solid acid-base catalyst is chosen from doped calcium hydroxyapatites.

In particular the molar ratio (Ca+M)/P of the calcium hydroxyapatite before doping (with Ca representing calcium, P representing phosphorus and M representing a metal) is from 1.5 to 2, preferably from 1.5 to 1.8, and preferentially from 1.6 to 1.8. According to the invention, M may represent a metal, a metal oxide or a mixture thereof, ranging from 0.1 mol % to 50 mol % of calcium substitution, preferably from 0.2 mol % to 20 mol %, M preferentially being chosen from Li, Na and K.

According to one embodiment, the solid acid-base catalyst is doped with one or more transition metals, more preferentially with transition metals chosen from the metals Ni, Co, Cu, Pd, Pt, Rh and Ru. According to the invention, the metals may be used alone or as a mixture. Preferably, said solid acid-base catalyst is doped with nickel.

According to the invention, the doping may take place via methods known to those skilled in the art, for instance by coprecipitation during the synthesis of the catalyst or by impregnation onto the solid acid-based catalyst before doping, preferentially onto an already-synthesized hydroxyapatite, of at least one precursor of said dopant, preferentially of said transition metal. The content of dopant, preferentially of transition metal, may be adapted by a person skilled in the art, but it is generally from 0.5% to 20% by weight, preferably from 1% to 10% by weight and preferentially from 1% to 5% by weight relative to the weight of the doped solid catalyst.

According to the invention, the doped solid catalyst may be calcined and at least partially reduced, to obtain, at least partly at the surface of the doped solid catalyst, the transition metal in an oxidation state of zero.

According to a particular embodiment, when the catalyst is doped with nickel, calcined and at least partially reduced, it has at least partly at its surface, nickel in an oxidation state of zero.

According to the invention, the oligomerization reaction is a catalytic reaction of heterogeneous type, insofar as it is performed in the gas phase and in the presence of a doped solid acid-base catalyst.

According to the invention, the oligomerization and especially the dimerization reaction may be performed at a pressure from 0.1 to 20 bar absolute (1 bar=10⁵ Pa), preferably from 0.3 to 15 bar absolute, preferentially from 0.5 to 10 bar absolute and more preferentially from 1 to 5 bar absolute.

In the oligomerization and especially the dimerization reaction of the process of the invention, one or more alcohols (Ai), especially ethanol, may be fed continuously as vapor phase. The flow rate of alcohol(s) (Ai) of said reaction may be from 1 to 8, preferably from 1 to 6 and preferentially from 1 to 5 g of alcohol (Ai) per hour and per g of doped solid acid-base catalyst.

According to the invention, the molar ratio between the hydrogen and the alcohol(s) (Ai) may be from 0.5 to 10, preferably from 1 to 8 and preferentially from 2 to 6. The hydrogen used for performing the process according to the invention may be used in pure form or diluted in an inert gas, such as nitrogen. In the case of dilution of the hydrogen, the amount of hydrogen present in said inert gas is such that it represents from 10% to 99% by volume of the hydrogen/inert gas mixture.

In the context of the invention, and unless otherwise mentioned, the term “production efficiency” means the measurement of the efficacy of the process. The production efficiency according to the invention corresponds to the amount of an alcohol (Aj), especially of butanol, produced per hour, for one gram of catalyst used in the process.

In the context of the invention, and unless otherwise mentioned, the term “yield” means the ratio, expressed as a percentage, between the obtained amount of product and the desired theoretical amount.

In the context of the invention, and unless otherwise mentioned, the term “selectivity” means the number of moles of alcohol (Ai), and especially of ethanol, transformed into desired product relative to the number of moles of alcohol (Ai) transformed.

In accordance with the process according to the invention, the gas-phase oligomerization and especially dimerization reaction, in the presence of hydrogen, may be performed using any reactor generally known to those skilled in the art.

According to one embodiment, the reaction is advantageously performed in a tubular or multitubular fixed-bed reactor, functioning in isothermal or adiabatic mode. It may also be performed in a catalyst-coated exchange reactor.

According to the invention, the doped solid acid-base catalyst is preferentially immobilized in a reactor in the form of grains or extrudates or supported on a metal foam.

According to the invention, the process consists of an oligomerization reaction of at least one alcohol (Ai) performed in the presence of hydrogen, which allows hydrogenation of the products derived from the oligomerization. Thus, the process according to the invention advantageously makes it possible to perform two successive reactions in a single step, without isolation of the intermediate species. Thus, the process according to the invention advantageously allows the use of only one piece of equipment, namely only one reactor and only one catalyst, to perform both the oligomerization and hydrogenation reaction in a single step.

According to the invention, after the reaction, a mixture (M′) is obtained, comprising at least one alcohol (Aj).

According to a particular embodiment, the process comprises a step of condensing the mixture (M′), after the oligomerization reaction, so as to obtain the mixture (M), said mixture (M) comprising at least one alcohol (Aj).

In the context of the invention, and unless otherwise mentioned, the term “mixture (M′)” means a mixture derived from the gas-phase oligomerization reaction of at least one alcohol (Ai), in the presence of hydrogen. The mixture (M′) thus represents a mixture that is gaseous at the reaction temperature.

In the context of the invention, and unless otherwise mentioned, the term “mixture (M)” means a mixture (M′) which has undergone a condensation step after the reaction. The mixture (M) thus represents a liquid mixture.

According to a particular embodiment, the mixture (M′) obtained after the gas-phase oligomerization reaction, in the presence of hydrogen, may be cooled to a temperature from 0° C. to 100° C., so as to condense the gaseous mixture (M′) to a liquid mixture (M).

According to the invention, the mixture (M) may comprise the remainder of unconverted alcohol(s) (Ai), and especially of ethanol, and water derived from the reaction and/or originating from new alcohol(s) (Ai), and alcohols (Aj), especially butanol.

According to a particular embodiment, the mixture (M) obtained according to the process may comprise at least 5% (by weight relative to the total weight of the mixture (M)) of butanol, and preferably at least 8% and preferentially at least 10% of butanol.

In the context of the invention, and unless otherwise mentioned, the term “new alcohol (Ai)” means the alcohol (Ai) used as starting reagent in the oligomerization reaction.

According to one embodiment, the remainder of unconverted alcohol(s) (Ai) may be recycled.

In the context of the invention, and unless otherwise mentioned, the term “recycling alcohol (Ai)” means the remainder of alcohol (Ai) not converted in the oligomerization reaction.

According to the invention, the new alcohol (Ai) differs from the recycling alcohol (Ai).

In accordance with the process according to the invention, said mixture (M) preferentially comprises several alcohols (Aj) whose linear or branched alkyl chain comprises m carbon atoms, with m representing an integer from 2 to 20. Preferably, said mixture (M) comprises at least butanol (m=4). According to another aspect of the invention, the mixture (M) comprises, besides butanol, other alcohols (Aj) whose linear or branched alkyl chain comprises m carbon atoms, with m representing an integer from 2 to 20. More particularly, the mixture (M) may comprise, besides butanol, linear alcohols, such as hexanol, pentanol, heptanol, octanol or decanol, or branched alcohols such as ethyl-2-butanol or ethyl-2-hexanol.

According to one aspect of the invention, the process may comprise, after the oligomerization and especially the dimerization reaction, and the condensation step, successive distillation steps to separate the various upgradable alcohols (Aj) from the mixture (M), and also steps for recycling alcohol(s) (Ai), especially ethanol.

More particularly, the mixture (M) containing the remainder of unconverted alcohol(s) (Ai), especially ethanol, the water derived from the reaction and/or originating from new alcohol(s) (Ai), and the upgradable alcohols, may be separated in a set of distillation columns intended for recovering the upgradable alcohols, removing the water derived from the reaction and the water derived from new alcohol(s) (Ai) (in the case where the alcohol(s) (Ai) used for the oligomerization are aqueous) and optionally recycling the unconverted alcohol(s) (Ai) of the reaction, generally in their azeotropic form.

According to the invention, the oligomerization and especially dimerization reaction, in the presence of hydrogen, may be performed at atmospheric pressure or under pressure.

According to one embodiment, in the case where the reaction is performed under pressure, the mixture (M) derived from the reaction may be depressurized to a pressure making it possible to perform the separation of the water/alcohol(s) (Ai) azeotrope and of the upgradable alcohols.

In the context of the invention, and unless otherwise mentioned, the term “depressurized mixture (M)” means a mixture (M) which has been depressurized after the oligomerization reaction, when the reaction is performed under pressure.

According to the invention, the mixture (M), optionally depressurized, derived from the process, may be directed to a set of two distillation columns denoted C1 and C2, fitted together to obtain three streams:
F1: the water/alcohol(s) (Ai) azeotrope, and especially the water/ethanol azeotrope, which is recycled;
F2: the water derived from new alcohol(s) (Ai) and also the water derived from the reaction; and
F3: the alcohols (Aj), especially butanol.

According to one embodiment, the columns C1 and C2 may be columns with plates or columns with packing.

The presence of the water/alcohol(s) (Ai) azeotrope, and especially the water/ethanol azeotrope, makes it difficult to remove the water from the reaction. To facilitate this separation, the phenomenon of demixing of the alcohol(s) (Aj)/water mixtures may be used. During the distillation to obtain the alcohols (Aj) (F3) at the bottom and the water/alcohol(s) (Ai) (F1) azeotrope at the top, demixing may take place to generate two liquid phases in equilibrium, a phase rich in alcohol(s) (Aj) and a phase rich in water. This phenomenon may be used to facilitate the separation of various constituents.

The feed may be performed in column C1, at the stage allowing the performance qualities of the assembly to be optimized.

According to the invention, a decanter may be installed at the bottom of column C1, below the feed plate which separates these two liquid phases, or the decanter may be installed inside or outside the column C1. The organic phase, rich in alcohol(s) (Aj), may be recycled as an internal reflux of the column Cl and makes it possible to obtain the mixture of alcohols (Aj) at the bottom of this column C1. The aqueous phase may leave the column C1 and be sent to a column C2 which may be a reflux separation column or a simple stripper. This column C2 may be boiled and may make it possible to obtain at the bottom a stream of water free of alcohols (Ai) and (Aj), and especially free of ethanol and butanol.

According to the invention, the distillate from the column C2 may preferentially be in vapor form, this column functioning at the same pressure as the column C1. The vapor phase of this column C2 may be sent to the column C1, preferentially to the stage above the stage of the liquid/liquid decanter. The top of the column C1 is standard and may comprise a condenser for obtaining the reflux necessary for the separation. The water/alcohol(s) (Ai) (F1) azeotrope, and especially the water/ethanol azeotrope, may then be obtained at the top. It may be obtained as a vapor phase or as a liquid phase. If it is obtained as a vapor phase, this avoids having to vaporize it before feeding the synthesis reaction, which advantageously makes it possible to reduce the necessary energy consumption.

According to the invention, the alcohols (Aj) (F3) are obtained at the bottom of the column C1. They may be separated by simple distillation in an additional column C3 in order to obtain pure butanol at the top and the other alcohols (Aj) other than butanol at the bottom.

The various alcohols (Aj) may then be separated via successive distillations to obtain these various alcohols in the order of their boiling points.

According to one embodiment, the new alcohol (Ai), and especially the new ethanol, which is pure or containing water and also optionally the recycling alcohol (Ai), especially the recycling ethanol, if it is liquid, may be vaporized and then superheated to the reaction temperature before entering a reactor in which the oligomerization takes place (oligomerization reactor). If the recycling alcohol (Ai), especially the recycling ethanol, is in vapor form, the new alcohol (Ai), and especially the new ethanol, may be vaporized and then superheated to the reaction temperature before entering the oligomerization reactor.

The process according to the invention advantageously allows the formation of desired alcohols in a single step, unlike the standard route using undoped hydroxyapatites, and comprising a dimerization reaction followed by a hydrogenation as described in patent application EP 2 206 763. The process according to the invention allows the use of a single catalyst and of a single reactor. It results therefrom that the process according to the invention advantageously allows a saving in space devoted to the equipment, and also a saving in time and in consequent facility.

The process according to the invention advantageously makes it possible to work at much lower temperatures than in a standard dimerization performed with undoped hydroxyapatites, i.e. at approximately 195° C., instead of approximately 400° C. There is a consequent saving in energy for an industrial process. This also makes it possible to limit the side reactions, which reduce the yields, which may take place in the gas phase at 400° C. Thus, the process according to the invention advantageously makes it possible, for example, to prevent the formation of aromatic compounds such as xylene or benzene which are formed in the gas phase at temperatures of 400° C. Now, these products are difficult to separate from ethanol and butanol. Avoiding their formation facilitates the post-reaction separations, which is an advantage from an industrial viewpoint.

Furthermore, the process according to the invention advantageously allows better selectivity. Specifically, doping with metals allows a decrease in the number of species present, especially intermediate species of alcohologen type, such as crotonyl alcohols (cis and trans), butanal, 1-butenol, hexanal, crotonaldehydes (cis and trans) which are found using undoped hydroxyapatites at 400° C. Moreover, the process according to the invention is advantageous since it makes it possible to stabilize the mixture over time, due to the absence of these aldehyde species.

Thus, the process according to the invention makes it possible to simplify the distillation of the crude reaction product, since the number of products to be separated is less than that of a case without doping. Specifically, the main reaction without doping generates components with a boiling point close to that of butanol, such as 2-buten-1-ol or 3-buten-1-ol, or components which form azeotropes with butanol or with each other, making separation difficult or even impossible.

Moreover, since the process according to the invention makes it possible to be rid of undesirable intermediate species, it can advantageously increase the selectivities and the yields of upgradable alcohols, and especially of butanol. Thus, the process according to the invention makes it possible to improve the efficacy and the overall selectivity of the process.

The examples that follow illustrate the invention without, however, limiting it.

### EXAMPLES

### Example 1

### Synthesis of an HAP Catalyst Doped With 7.5% (by Weight) of Nickel

A nickel solution was prepared by adding 44.8 g of Ni(NO₃)₂.6H₂O to a graduated flask and then making up the volume to 50 ml with demineralized water. 9 ml of this solution were then added slowly, using a syringe, to 20 g of hydroxyapatite (Ca/P ratio=1.67) (supplier: Sangi), in a stirred round-bottomed flask. Stirring was maintained for 30 minutes. The solid was then dried in a muffle furnace at 120° C. for 2 hours, and the solid was then calcined at 450° C. for 2 hours in air, and the solid was finally allowed to return to room temperature. The catalyst thus obtained contains 7.5% by weight of nickel.

### Example 2

### Synthesis of an HAP Catalyst Doped With 1% (by Weight) of Nickel

A nickel solution was prepared by adding 5.55 g of Ni(NO₃)₂.6H₂O to a graduated flask and then making up the volume to 50 ml with demineralized water. 9 ml of this solution were then added slowly, using a syringe, to 20 g of hydroxyapatite (Ca/P ratio=1.67) (supplier: Sangi), in a stirred round-bottomed flask. Stirring was maintained for 30 minutes. The solid was then dried in a muffle furnace at 120° C. for 2 hours, and the solid was then calcined at 450° C. for 2 hours in air, and was finally allowed to return to room temperature. The catalyst thus obtained contains 1% by weight of nickel.

### Example 3

### Reaction Performed at 195° C. With an HAP Doped With 7.5% by Weight of Nickel

6 g of catalyst derived from Example 1 were placed in a glass reactor (22 mm in diameter and 20 cm tall) between 7.5 ml (below) and 17 ml (above) of glass powder (300-600 μm). A stream of nitrogen and hydrogen was then circulated in the reactor at room temperature for 30 minutes. The reactor was then heated at 400° C. for 2 hours, and was then placed at 195° C. Only a hydrogen flow rate of 350 ml/minute was left. The reaction was performed at atmospheric pressure (P=1 bar). 95% ethanol (water qs 100) was then added, using a syringe plunger, to the reactor at 195° C., at a flow rate of 13.5 ml/hour, corresponding to a hydrogen/ethanol molar ratio of 4. A liquid phase was recovered at the reactor outlet by cooling the collecting flask with cardice. The mixture obtained was injected into a gas chromatograph (GC Agilent HP6890N, HP-innowax (PEG) 30 m×0.25 mm×0.25 μm column, FID detector, cyclohexanol internal standard) for analysis.

The conversion into ethanol is 11.2% and the weight percentages of the various products are as follows:
Butanol: 3.82% (42.6% selectivity)
Acetaldehyde: 0.48%
1-Butenol: 0%
Crotonyl alcohol: 0%
Diethyl ether: 0%
Butadiene: 0%
Butanal: 0%
Ethylbutanol: 0.21%
Hexanol: 0.46%
Hexanal: 0%
Ethylhexanol: 0.1%
Octanol: 0.08%
Xylene: 0%

Thus, a butanol yield of 4.8% and a production efficiency of 0.065 g of butanol per hour and per g of catalyst were obtained. Thus, it was observed that the species formed under these conditions are only alcohols.

### Example 4 (Comparative Example)

### Reaction Performed at 195° C. With an Undoped HAP

This example corresponds to amounts identical to those of Example 3, but with an undoped catalyst.

6 g of hydroxyapatite catalyst with a Ca/P ratio of 1.67 (supplier: Sangi) were placed in a glass reactor (22 mm in diameter and 20 cm tall) between 7.5 ml (below) and 17 ml (above) of glass powder (300-600 μm). A stream of nitrogen and hydrogen was then circulated in the reactor at room temperature for 30 minutes. The reactor was then heated at 400° C. for 2 hours, and was then placed at 195° C. Only a hydrogen flow rate of 350 ml/minute was left. The reaction was performed at atmospheric pressure (P=1 bar). 95% ethanol (water qs 100) was then added, using a syringe plunger, to the reactor at 195° C., at a flow rate of 13.5 ml/hour, corresponding to a hydrogen/ethanol molar ratio of 4. A liquid phase was recovered at the reactor outlet by cooling the collecting flask with cardice. The mixture obtained was injected into a gas chromatograph (GC Agilent HP6890N, HP-innowax (PEG) 30 m×0.25 mm×0.25 μm column, FID detector, cyclohexanol internal standard) for analysis.

It was observed that the ethanol was not converted and that no trace of butanol or of other alcohols was detected.

### Example 5

### Reaction Performed at 195° C. With an HAP Doped With 1% by Weight of Nickel

6 g of catalyst derived from Example 2 were placed in a glass reactor (22 mm in diameter and 20 cm tall) between 7.5 ml (below) and 17 ml (above) of glass powder (300-600 μm). A stream of nitrogen and hydrogen was circulated in the reactor at room temperature for 30 minutes. The reactor was then heated at 400° C. for 2 hours, and then placed at 195° C. Only a hydrogen flow rate of 350 ml/minute was left. The reaction was performed at atmospheric pressure (P=1 bar). 95% ethanol (water qs 100) was then added, using a syringe plunger, to the reactor at 195° C., at a flow rate of 13.5 ml/hour, corresponding to a hydrogen/ethanol molar ratio of 4. A liquid phase was recovered at the reactor outlet by cooling the collecting flask with cardice. The mixture obtained was injected into a gas chromatograph (GC Agilent HP6890N, HP-innowax (PEG) 30 m×0.25 mm×0.25 μm column, FID detector, cyclohexanol internal standard) for analysis.

The conversion into ethanol is 9.4% and the weight percentages of the various products are as follows:
Butanol: 4.21% (56.2% selectivity)
Acetaldehyde: 0.31%
1-Butenol: 0%
Crotonyl alcohol: 0%
Diethyl ether: 0%
Butadiene: 0%
Butanal: 0%
Ethylbutanol: 0.29%
Hexanol: 1%
Hexanal: 0%
Ethylhexanol: 0.12%
Octanol: 0.165%
Xylene: 0%

A butanol yield of 5.3% and a production efficiency of 0.073 g of butanol per hour and per g of catalyst were obtained. Thus, it was observed that the species formed under these conditions are virtually only alcohols. The selectivity toward alcohol was improved by using lower doping with nickel. A selectivity toward alcohols of 75% was obtained.

### Example 6 (Comparative Example)

### Reaction Performed at 250° C. With a Hydroxyapatite Doped With 1% by Weight of Nickel

This example corresponds to amounts identical to those of Example 5, but with a temperature of 250° C.

6 g of catalyst derived from Example 2 were placed in a glass reactor (22 mm in diameter and 20 cm tall) between 7.5 ml (below) and 17 ml (above) of glass powder (300-600 μm). A stream of nitrogen and hydrogen was circulated in the reactor at room temperature for 30 minutes. The reactor was then heated at 400° C. for 2 hours, and then placed at 250° C. Only a hydrogen flow rate of 350 ml/minute was left. The reaction was performed at atmospheric pressure (P=1 bar). 95% ethanol (water qs 100) was then added, using a syringe plunger, to the reactor at 250° C., at a flow rate of 13.5 ml/hour, corresponding to a hydrogen/ethanol molar ratio of 4. A liquid phase was recovered at the reactor outlet by cooling the collecting flask with cardice. The mixture obtained was injected into a gas chromatograph (GC Agilent HP6890N, HP-innowax (PEG) 30 m×0.25 mm×0.25 μm column, FID detector, cyclohexanol internal standard) for analysis.

The conversion into ethanol is 57.7% and the weight percentages of the various products are as follows:
Butanol: 0%
Acetaldehyde: 2.9%
Acetal: 6%
1-Butenol: 0%
Crotonyl alcohol: 0%
Diethyl ether: 0%
Butadiene: 0.15%
Butanal: 0%
Ethylbutanol: 0.68%
Hexanol: 1.23%
Hexanal: 0.3%
Ethylhexanol: 0.27%
Octanol: 0.27%
Xylene: 0%

Thus, at the end of the reaction, a butenol yield of 0% was obtained. Consequently, the reaction does not function at a temperature as high as 250° C.

### Example 7 (Comparative Example)

### Reaction Performed at 400° C. With an Undoped Hydroxyapatite

6 g of hydroxyapatite catalyst with a Ca/P ratio of 1.67 (supplier: Sangi) were placed in a glass reactor (22 mm in diameter and 20 cm tall) between 7.5 ml (below) and 17 ml (above) of glass powder (300-600 μm). The reactor was placed at 400° C. and 95% (by weight) ethanol was then added as a gaseous phase at a flow rate of 28.2 ml/hour with a hydrogen flow rate of 288 ml/minute. The reaction was performed at atmospheric pressure (P=1 bar). A liquid phase was recovered at the reactor outlet by cooling the collecting flask with cardice. The mixture obtained was injected into a gas chromatograph for analysis.

The conversion into ethanol is 28.7% and the weight percentages of the various products are as follows:
Butanol: 7.1%
Crotonyl alcohols: 0.7%
1-Butenol: 0.2%
Acetaldehyde: 0.2%
Acetal: 0.05%
Diethyl ether: 0.05%
Ethyl butyl ether: 0.02%
Butadiene: 0.6%
Butanal: 0.2%
Hexanol: 0.7%
Ethylbutanol: 0.6%
Hexanal: 0.05%
Ethylhexanol: 0.2%
Octanol: 0.1%
Decanol: 0.02%
Xylene: 0.06%
Ethylene: 0.1%
Butene: 0.02%
Hexene: 0.01%
Hexadiene: 0.2%
Benzene: 0.1%

When the reaction is performed at 400° C. with an undoped hydroxyapatite in the presence of hydrogen, the range of products obtained is very broad and contains undesirable species such as alkenes and aromatic compounds.

1. A process for preparing a mixture (M) comprising at least one alcohol (Aj), said process comprising a gas-phase oligomerization reaction of at least one alcohol (Ai), performed in the presence of hydrogen, and of a solid acid-base catalyst doped with one or more metals, at a temperature of greater than or equal to 50° C. and strictly less than 200° C.

2. The process as claimed in claim 1, wherein said temperature is from 80° C. to 195° C.

3. The process as claimed in claim 1, wherein said oligomerization reaction is a dimerization of ethanol.

4. The process as claimed in claim 1, wherein said mixture (M) comprises butanol.

5. The process as claimed in claim 1, wherein said mixture (M) comprises several alcohols (Aj) whose linear or branched alkyl chain comprises m carbon atoms, with m representing an integer from 2 to 20.

6. The process as claimed in claim 1, wherein said solid acid-base catalyst before doping is selected from the group consisting of:
alkaline-earth metal phosphates;
hydrotalcites;
zeolites; and
mixtures of metal oxides.

7. The process as claimed in claim 1, wherein said solid acid-base catalyst before doping is selected from the group consisting of calcium hydroxyapatites.

8. The process as claimed in claim 7, wherein said calcium hydroxyapatite has a (Ca+M)/P molar ratio from 1.5 to 2, M being a metal, a metal oxide or a mixture thereof.

9. The process as claimed in claim 1, wherein said solid acid-base catalyst is doped with one or more transition metals.

10. The process as claimed in claim 9, wherein said one or more transition metals are selected from the group consisting of Ni, Co, Cu, Pd, Pt, Rh and Ru.

11. The process as claimed in claim 1, wherein said doped solid acid-base catalyst is immobilized in a reactor in the form of grains or extrudates or supported on a metal foam.

12. The process as claimed in claim 1, wherein said oligomerization reaction is performed in a tubular or multitubular fixed bed reactor, functioning in isothermal or adiabatic mode.

13. The process as claimed in claim 1, wherein said oligomerization reaction is performed at a pressure from 0.1 to 20 bar absolute.

14. The process as claimed in claim 1, wherein said at least one alcohol (Ai) has a flow rate from 1 to 8 g of said at least one alcohol (Ai), per hour and per g of said doped solid acid-base catalyst.

15. The process as claimed in claim 1, wherein a molar ratio between said hydrogen and said at least one alcohol (Ai) from 0.5 to 10 is used.

16. The process as claimed in claim 1, further comprising a condensation step after said oligomerization reaction, to obtain said mixture (M).

17. The process as claimed in claim 1, wherein said mixture (M) is subjected to successive distillation steps to separate the one or more alcohols (Aj) from said mixture (M), and also steps for recycling said at least one alcohol (Ai).

18. The process as claimed in claim 1, wherein said solid acid-base catalyst before doping is selected from the group consisting of tricalcium phosphates, calcium hydrogen phosphates, and calcium hydroxyapatites.
